(19) United States
(12) Patent Application Publication
(10) Pub. No.: US 2010/0298915 A1
(43) Pub. Date: Nov. 25, 2010

(54) COMBINATION TENSION WRAP

(76) Inventor: Jeffrey Thomas Whitely, Millgrove (CA)

Correspondence Address: F. CHAU & ASSOCIATES, LLC, 130 WOODBURY ROAD, WOODBURY, NY, 11797, US

(21) Appl. No.: 12/668,616

(22) PCT Filed: May 27, 2008
(86) PCT No.: PCT/CA08/01020

§ 371 Date: Jan. 11, 2010

(30) Foreign Application Priority Data
Jul. 13, 2007 (CA) 2593678

(51) Int. Cl. A61F 7/03; A61F 7/02

(52) U.S. Cl. 607/112

(57) ABSTRACT

A thermotherapeutic pad has an envelope containing a thermally active material attached to a flexible flap which can be wrapped a body part and fastened in position.

### FIELD OF THE INVENTION

This invention relates to thermotherapeutic pads.

### BACKGROUND OF THE INVENTION

Thermotherapeutic pads are used to apply heat or cold to a part of the body. Depending upon the nature of the injury or discomfort, heat and/or cold may have a beneficial therapeutic effect and may also provide some relief from discomfort to the user.

Thermotherapeutic pads may be reusable, for example providing an envelope containing a material that can be repeatedly heated in a conventional oven or microwave oven, or cooled in a refrigerator or freezer, or both. The thermotherapeutic pad remains flexible when hot or cold, and thus can be applied to the skin and conform to the body part to provide a broad area of surface contact between pad and the skin, and thereby effectively apply heat or cold to the affected area.

Thermotherapeutic pads may alternatively be disposable after a one-time use, for example providing an outer envelope containing a combination of chemicals which are kept separated until the thermotherapeutic pad is ready for use, for example by maintaining one of the chemicals in a frangible ampoule or breakable envelope inside the outer envelope that can be ruptured upon the application of pressure to allow the chemicals to mix. The mixing of the chemicals results in an endothermic reaction in the case of a cold pack, so that the thermotherapeutic pad draws heat from the skin, or an exothermic endothermic in the case of a heating pad, so that the thermotherapeutic pad applies heat to the skin.

The user must use at least one hand to hold the thermotherapeutic pad against the part of the body desired to be treated for a prolonged interval, for example several minutes. It can be uncomfortable to maintain the thermotherapeutic pad in the same position for this length of time, and the user's hand may become overly sensitive to the cooling or heating effect of the thermotherapeutic pad after prolonged contact. Moreover, although the thermotherapeutic pad has a fairly large surface area for contacting the skin, when manually held against the skin it is difficult to ensure that the entire surface area of the pad is used, so the pad may not transfer heat efficiently to or from the tissue. Applying pressure or compression to a party of the body may also prevent or reduce swelling by restricting the blood flow.

### BRIEF DESCRIPTION OF THE DRAWINGS

In drawings which illustrate by way of example only a preferred embodiment of the invention,

FIG. 1 is a perspective view of a thermotherapeutic pad according to the invention.

FIG. 2 is a perspective view showing the thermotherapeutic pad of FIG. 1 in use.

FIG. 3 is a cross-sectional view of the thermotherapeutic pad in FIG. 2.

FIG. 4 is a partly cutaway perspective view of the thermotherapeutic pad of FIG. 1.

### DETAILED DESCRIPTION OF THE INVENTION

The invention thus provides a thermotherapeutic pad comprising a pack portion comprising an envelope containing a thermally active material; and a flexible flap affixed to the pack portion for wrapping a body part, the flap comprising a fastener, whereby the pack portion can be placed against the body part and the flap wrapped around the body part and fastened to the pack portion or to the flap to maintain the pack portion in position against the body part.

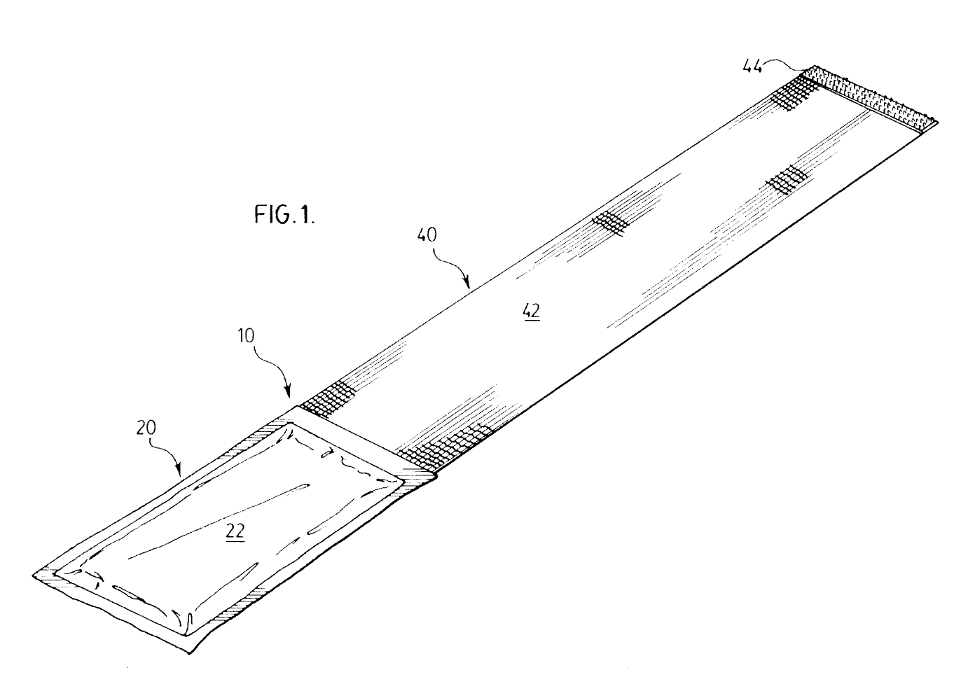

An embodiment of a thermotherapeutic pad according to the invention as illustrated in FIG. 1. The pad 10 comprises a pack portion 20 and a wrap portion 40.

The pack portion 20 comprises an envelope 22 containing a thermally active material (or combination of materials) 24. In the embodiment shown the pack portion 20 is a disposable ‘cold pack,’ by way of example, so the thermally active material 24 is a mixture of chemicals which causes an endothermic reaction and the envelope 22 is composed at least in part of a fluid-tight material which is sealed to contain the chemical mixture. The envelope 22 is preferably flexible, allowing the pack portion 20 to conform to the body part against which it is placed, as is well known to those skilled in the art.

The invention will be described in the context of the disposable cold pack illustrated, however the structure and principles of the invention apply equally to a reusable cold pack (using for example a flexible gel which remains pliant at temperatures below the freezing temperature of water), or a heating pad containing a material which absorbs heat and preferably has a high thermal inertia so as to release the heat slowly over a period of time, as is well known.

According to the invention, a wrap 40 comprising a flexible flap 42 is affixed to the pack portion 20, preferably at one end. The flap 42 is preferably (but not necessarily) elastic to facilitate the application of a compressive force when the pad 10 is in use, and may be composed of a porous fabric which ‘breathes,’ i.e. is permeable to air, to minimize discomfort to the user. The flap 42 may be affixed to the envelope 22 by sealing an end of the flap between envelope layers, as at 24 in FIG. 4, using any suitable technique, fastening member and/or adhesive.

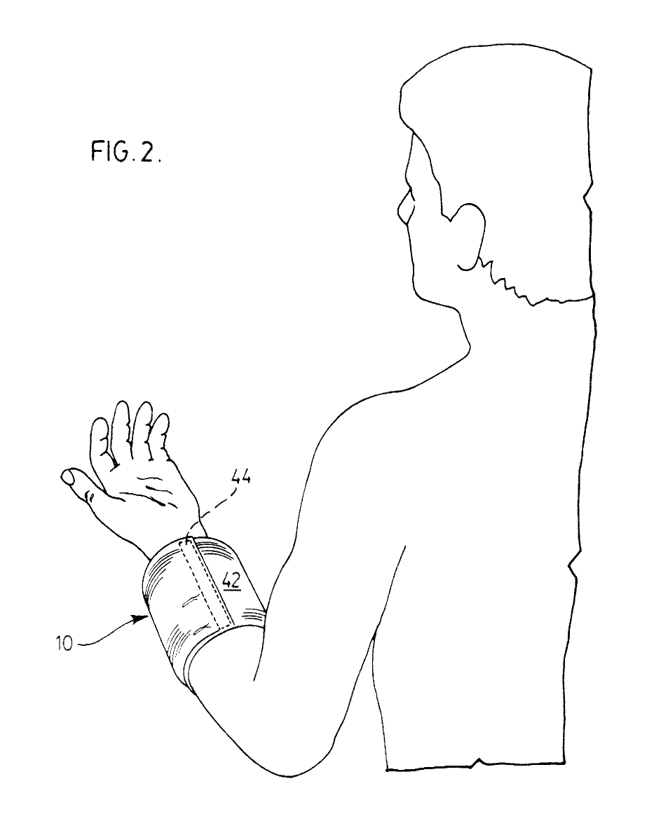

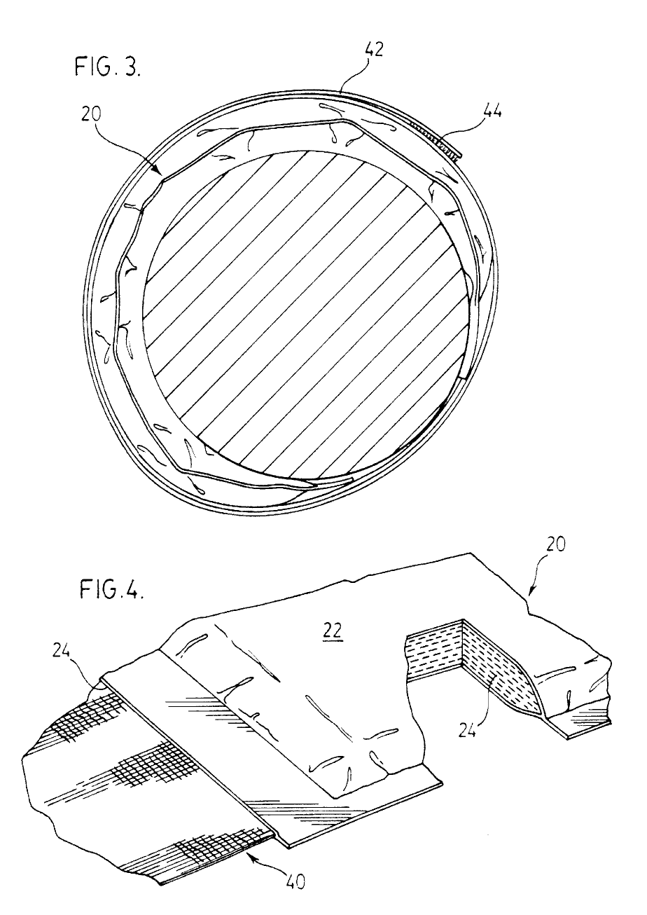

In the embodiment shown, the flap 42 wraps around both the pack portion 20 and the body part. The flap 42 may alternatively be fastened to any convenient portion of the exterior of the pack portion 20 by suitable means. The length of the flap may be selected according to the intended use; for example a thermotherapeutic pad 10 intended for use on the user's back could have a longer flap 42 than a pad 10 designed for use on a limb such as that illustrated in FIGS. 2 and 3. In embodiments in which the flap 42 wraps around the pack portion 20, the flap may be composed of a thermally insulating material which will insulate the pack portion 20 from ambient air and thus reduce the rate of change of temperature of the pack portion 20.

The flap 42 is provided with a fastener 44, preferably releasable, for example the hook portion of a hook and loop type fastener such as VELCRO™ which can be releasably affixed to any portion of the porous material of the flap 42, maximizing the number of positions in which the flap can be affixed to thus accommodate the wide variety of sizes and shapes of body parts to which the pad 10 may be applied, for example as shown in FIGS. 2 and 3. As shown, the fastener 44 may conveniently be a strip of fastener fabric traversing the width of the flap 42 at the free end of the flap 42.

Alternatively, if the flap material is such that a hook strip 44 does not readily attach it, a complementary loop fabric (not shown) may be attached to the flap 42 at one or more convenient locations to provide a releasable attachment point for the a hook strip 44. This is a particularly convenient fastening arrangement, but other suitable fastening means may be used. For example, a complementary loop fabric (not shown) may be attached to the pack portion 20 at one or more convenient locations to provide a releasable attachment point for the hook strip 44.

To use the disposable thermotherapeutic pad or ‘cold pack’ illustrated, the ampoule or inner envelope is broken by the application of force, allowing the chemicals to mix in the pack portion 20 and the endothermic reaction to commence. The pack portion 20 is placed over the affected body part, for example the forearm as illustrated in FIG. 2, and the flap 42 is wrapped around the body part and the pack 20 to maintain the pack portion 20 in position against the skin. The flap 42 may also be used to apply compression to the body part. The fastening strip 44 is attached to the flap material once the desired tension has been reached.

The flap 42 applies a circumferential pressure to the pack portion 20, allowing the pack portion 20 to contact the skin over substantially the entire surface area of the pad and thereby increasing the effectiveness of heat transfer between the pack portion 20 and the body part. The flap 42 also keeps the pack portion 20 from shifting its position once in place.

Alternatively in the case of a disposable thermotherapeutic pad 10 intended for one-time use, the fastener 44 does not need to be releasable. The flap 42 can be cut to remove the thermotherapeutic pad 10 after use.

An embodiment of the invention for a reusable cold pack can be placed in the freezer to chill the flexible gel (or other coolable material) prior to use. In this embodiment the flap 42 is preferably formed from a water resistant material, so that prolonged or repeated storage in a freezer does not cause the material to become unduly damp or soggy.

An embodiment of the invention with a disposable heating pad utilizing chemicals that react exothermically operates in substantially the same fashion as the embodiment described in respect of the disposable thermotherapeutic pad 10 illustrated. An embodiment of the invention utilizing a reusable heating pad, which may for example contain oats or another suitable material that can be heated in a conventional oven or a microwave oven, utilizes a material for the flap 42 which will not be damaged or lose it's flexibility when heated to the desired temperature.

Various embodiments of the present invention having been thus described in detail by way of example, it will be apparent to those skilled in the art that variations and modifications may be made without departing from the invention.

1-20. (canceled)

21. A thermotherapeutic pad comprising
a pack portion comprising two layers sealed together to form a closed envelope, the envelope having a seam along a side edge thereof, the pack portion further comprising thermally active material contained within the closed envelope; and
a flexible flap, for wrapping a body part, affixed to the pack portion along the seam, the flap comprising a fastener, whereby the pack portion can be placed against the body part and the flap wrapped around the body part and fastened to the pack portion or to the flap to maintain the pack portion in position against the body part.

22. The thermotherapeutic pad of claim 21 wherein the fastener comprises a hook material adapted to releasably fasten to a fabric.

23. The thermotherapeutic pad of claim 22 wherein the flap is composed of a fabric to which the hook material can releasably fasten.

24. The thermotherapeutic pad of claim 21 wherein the fastener is attached to a free end of the flap.

25. The thermotherapeutic pad of claim 21 wherein the flap is elastic.

26. The thermotherapeutic pad of claim 21 wherein the flap is affixed to the pack portion by adhesive to the seam of the envelope.

27. The thermotherapeutic pad of claim 21 wherein the strap is permeable to air.

28. The thermotherapeutic pad of claim 21 wherein the thermally active material comprises a mixture of chemicals which combine in an endothermic reaction.

29. The thermotherapeutic pad of claim 28 wherein the envelope comprises an inner rupturable envelope containing a first chemical mixture which combines in an endothermic reaction with the chemical mixture outside the inner rupturable envelope.

30. The thermotherapeutic pad of claim 21 wherein the thermally active material comprises a flexible gel with a high specific heat capacity for absorbing heat from the body part.

31. The thermotherapeutic pad of claim 21 wherein the thermally active material comprises a mixture of chemicals which combine in an exothermic reaction.

32. The thermotherapeutic pad of claim 31 wherein the envelope comprises an inner rupturable envelope containing a first chemical mixture which combines in an exothermic reaction with the chemical mixture outside the inner rupturable envelope.

33. The thermotherapeutic pad of claim 32 wherein the envelope contains a material with a high specific heat for transferring heat from the envelope to the body part.

34. A thermotherapeutic pad comprising
a pack portion comprising two layers sealed together to form a closed envelope, the envelope having a seam along a side edge thereof and containing a thermally active material; and
a flexible, elastic, air permeable flap for wrapping a body part, the flap being composed of a fabric that is affixed to the seam of the envelope by adhesive, the flap comprising a hook material attached to a free end of the flap and adapted to releasably fasten to the fabric,
whereby the pack portion can be placed against the body part and the flap wrapped around the body part and fastened to the pack portion or to the flap to maintain the pack portion in position against the body part.

35. The thermotherapeutic pad of claim 34, wherein the thermally active material is a mixture of chemicals which combine in an endothermic reaction.

36. The thermotherapeutic pad of claim 35, wherein the envelope comprises a first chemical mixture and an inner rupturable envelope containing a second chemical mixture, the second chemical mixture combining in an endothermic reaction with the first chemical mixture when the inner rupturable envelope is ruptured.

37. The thermotherapeutic pad of claim 34 wherein the thermally active material comprises a flexible gel with a high specific heat for absorbing heat from the body part.

38. The thermotherapeutic pad of claim 34 wherein the thermally active material comprises a mixture of chemicals which combine in an exothermic reaction.

39. The thermotherapeutic pad of claim 35, wherein the envelope comprises a first chemical mixture and an inner rupturable envelope containing a second chemical mixture, the second chemical mixture combining in an exothermic reaction with the first chemical mixture when the inner rupturable envelope is ruptured.

40. The thermotherapeutic pad of claim 39 wherein the envelope contains a material with a high specific heat capacity for transferring heat to a body part.
